(19) United States
(12) Patent Application Publication
(10) Pub. No.: US 2010/0030000 A1
(43) Pub. Date: Feb. 4, 2010

(54) PRODUCTION METHOD OF ALPHA-OLEFIN LOW POLYMER AND STORAGE METHOD OF PYRROLE COMPOUND

(75) Inventors: Hiroki Emoto, Okayama (JP); Kazuyuki Yokoyama, Okayama (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Mitsubishi Chemical Corporation, Tokyo (JP)

(21) Appl. No.: 12/519,525

(22) PCT Filed: Oct. 25, 2007
(86) PCT No.: PCT/JP07/70849

§ 371 Date: Jun. 17, 2009

(30) Foreign Application Priority Data
Dec. 28, 2006 (JP) 2006-354249

(51) Int. Cl. C07C 2/24

(52) U.S. Cl. 585/512

(57) ABSTRACT

The object of the present invention is to provide production method of an α-olefin low polymer using a chromium series catalyst comprising a pyrrole compound as a component. The present invention relates to that in producing an α-olefin low polymer such as 1-hexene using an α-olefin such as ethylene as a raw material, a chromium series catalyst constituted of a chromium compound (a), a pyrrole compound (b) and an aluminum-containing compound (c) is used as a polymerization catalyst, and a concentration of a pyrrole dimer contained in the pyrrole compound (b) is 2% by weight or less based on the pyrrole compound (b).

### TECHNICAL FIELD

The present invention relates to a production method of an α-olefin low polymer. More particularly, it relates to a production method of an α-olefin low polymer such as 1-hexene.

### BACKGROUND ART

Conventionally, a production method in which an α-olefin low polymer such as 1-hexene is selectively obtained using an α-olefin such as ethylene as a raw material and using a chromium series catalyst is known.

For example, Patent Document 1 reports a production method in which an α-olefin low polymer mainly comprising 1-hexene is obtained in high yield and high selectivity using a chromium series catalyst comprising a chromium compound, a nitrogen-containing compound such as an amine, an alkyl aluminum compound and a halogen-containing compound (see Patent Document 1 and Patent Document 2).

Patent Document 1: JP-A-08-003216

Patent Document 2: JP-A-10-109946

### DISCLOSURE OF THE INVENTION

### Problems that the Invention is to Solve

By the way, a chromium series catalyst is prepared by combining a chromium compound such as chromium (III)-2-ethylhexanoate with a nitrogen-containing compound such as an amine, and further adding other components.

Of the nitrogen-containing compounds used in combination with the chromium compound, a secondary amine is preferred, and pyrrole and pyrrole derivatives are particularly preferred.

However, it is known that pyrroles easily form polymers such as a dimer and a trimer. For this reason, a slight amount of those pyrrole polymers is often contained under the general storage conditions except for just after purification.

Such polymers contained in pyrroles have a low solubility particularly to a non-polar solvent such as hexene, heptane, cyclohexane or methylcyclohexane. Therefore, the polymers convert into substances having adhesion in a liquid such as a catalyst preparation liquid or a reaction liquid. Where catalyst components, a polymeric substances by-produced by low polymerization reaction of an α-olefin, deactivated catalyst components or the like are present as a solid in the liquid, the substances having adhesion incorporate those solids therein, and may adhere to a catalyst preparation tank, a wall of a reactor, a stirring machine, a nozzle and the like for obtaining an α-olefin low polymer. Formation of such deposits becomes a main cause that fluctuates a catalyst concentration in a reactor, or causes fouling of a reactor and the like, thereby making running operation such as heat removal unstable, in addition to economical loss due to catalyst loss.

The present invention has been made to solve technical problems in such a production method of an α-olefin low polymer.

Accordingly, an object of the present invention is to provide further industrially advantageous production method of an α-olefin in the production of an α-olefin low polymer using a chromium series catalyst containing a pyrrole compound.

### Means for Solving the Problems

As a result of extensive and intensive investigations to solve the above problems, the present inventors have reached to achieve the present invention. That is, the gist of the present invention resides in the following (1) to (6).

(1) A production method of an α-olefin low polymer, which comprises polymerizing an α-olefin in the presence of a chromium series catalyst, characterized in that:

the chromium series catalyst is constituted of at least a chromium compound (a), a pyrrole compound (b) and an aluminum-containing compound (c), and

a concentration of a pyrrole dimer contained in the pyrrole compound (b) in the chromium series catalyst that is applied to low polymerization reaction of an α-olefin is 2% by weight or less based on the pyrrole compound (b).

(2) The production method of an α-olefin low polymer described in claim 1, characterized in that the pyrrole compound (b) is sealed with an inert gas having an oxygen concentration of 1% or less after purification, and stored under light shielding.

(3) The production method of an α-olefin low polymer described in (1) or (2), characterized in that the pyrrole compound (b) is a pyrrole having one or plural alkyl groups having from 1 to 4 carbon atoms.

(4) The production method of an α-olefin low polymer described in any one of (1) to (3), characterized in that the chromium series catalyst is constituted of a combination of the chromium compound (a), the pyrrole compound (b), the aluminum-containing compound (c) and a halogen-containing compound (d).

(5) The production method of an α-olefin low polymer described in any one of (1) to (4), characterized in that the α-olefin is ethylene.

(6) The production method of an α-olefin low polymer described in any one of (1) to (5), characterized in that the α-olefin low polymer is 1-hexene.

(7) A storage method of a pyrrole compound, characterized in that a concentration of a pyrrole dimer in the pyrrole compound is 2% by weight or less based on the pyrrole compound.

(8) The storage method described in (7), characterized in that the pyrrole compound is purified, and then sealed with an inert gas having an oxygen concentration of 1% or less under light shielding.

Thus, according to the present invention, there is provided a production method of an α-olefin low polymer, which comprises polymerizing an α-olefin in the presence of a chromium series catalyst, characterized in that the chromium series catalyst is constituted of at least a chromium compound (a), a pyrrole compound (b) and an aluminum-containing compound (c), and a concentration of a pyrrole dimer contained in the pyrrole compound (b) in the chromium series catalyst that is applied to low polymerization reaction of an α-olefin is 2% by weight or less based on the pyrrole compound (b).

In the production method of an α-olefin low polymer to which the present invention is applied, the pyrrole compound (b) used as a component constituting the chromium series catalyst is preferably sealed with an inert gas having an oxygen concentration of 1% or less after purification, and then stored.

The pyrrole compound (b) is preferably stored in a stainless steel container or a carbon steel container under light shielding.

It is preferred that dimethylpyrrole having one or plural alkyl substituents having from 1 to 20 carbon atoms is used as the pyrrole compound (b).

The chromium series catalyst used in the production method of an α-olefin low polymer to which the present invention is applied is preferably constituted of a combination of the chromium compound (a), the pyrrole compound (b), the aluminum-containing compound (c) and a halogen-containing compound (d).

The α-olefin is preferably ethylene.

The α-olefin low polymer is preferably 1-hexene.

### ADVANTAGE OF THE INVENTION

According to the present invention, in the production of an α-olefin low polymer, deposits adhered to a catalyst preparation tank and a reactor are reduced, and running operation can be stabilized.

### BRIEF DESCRIPTION OF THE DRAWING

FIG. 1 is a view explaining a production flow example of an α-olefin low polymer in the embodiment of the invention.

### DESCRIPTION OF REFERENCE NUMERALS AND SIGNS

1c . . . Catalyst tank
10 . . . Reactor
10a . . . Stirring machine
11, 22, 32, 41, 42, 51 . . . Piping
11a . . . Deactivator supply piping
12 . . . First supply piping
12a . . . Ethylene supply piping
13 . . . Second supply piping
13a . . . Catalyst supply piping
14 . . . Third supply piping
15 . . . Fourth supply piping
21, 31 . . . Circulation piping
16 . . . Condenser
17 . . . . Compressor
20 . . . Degassing tank
30 . . . Ethylene separation column
40 . . . High boiling separation column
50 . . . Hexene separation column
52 . . . Solvent circulation piping
60 . . . Solvent drum

### BEST MODE FOR CARRYING OUT THE INVENTION

The best mode for carrying out the invention (hereinafter, the embodiment of the invention) is described in detail below. The invention is not limited to the following embodiment, and can be carried out with various modifications within a scope of its gist. Furthermore, the drawings used are to explain the present embodiment, and do not show the actual size.

(α-olefin)

In the production method of an α-olefin low polymer to which the embodiment of the invention is applied, the α-olefin used as a raw material includes substituted or unsubstituted α-olefins having from 2 to 30 carbon atoms. Specific examples of such an α-olefin include ethylene, propylene, 1-butene, 1-hexene, 1-octene, 3-methyl-1-butene and 4-methyl-1-pentene. In particular, ethylene is preferred as the α-olefin of a raw material, and when ethylene is used as the raw material, 1-hexene as a trimer of ethylene is obtained in high yield and high selectivity. Furthermore, when ethylene is used as the raw material, impurity components other than ethylene may be contained in the raw material. Specific impurity components include methane, ethane, acetylene and carbon dioxide. Those components are preferably in an amount of 0.1 mol % or less based on ethylene of the raw material.

### (Chromium Series Catalyst)

The chromium series catalyst is descried below. The chromium series catalyst used in the embodiment of the invention includes a catalyst constituted of a combination of at least a chromium compound (a), a pyrrole compound (b) and an aluminum-containing compound (c).

The chromium series catalyst used in the embodiment of the invention may contain a halogen-containing compound (d) as a fourth component according to need. Each component is described below.

### (Chromium Compound (a))

The chromium compound (a) used in the embodiment of the invention includes at least one compound represented by the general formula CrXₙ. In the general formula, X represents an optional organic group or inorganic group, or a negative atom, and n is an integer of from 1 to 6, and is preferably 2 or more. When n is 2 or more, X may be the same or different.

Examples of the organic group include a hydrocarbon group having from 1 to 30 carbon atoms, a carbonyl group, an alkoxy group, a carboxyl group, a β-diketonate group, a β-ketocarboxyl group, a β-ketoester group and an amido group.

Examples of the inorganic group include chromium salt-forming groups such as a nitric acid group or a sulfuric acid group. Examples of the negative atom include oxygen and a halogen. (Here, a halogen-containing chromium compound is not included in the halogen-containing compound (d) described hereinafter.)

The number of valency of chromium (Cr) is 0 to 6. The preferred chromium compound (a) includes a carboxylate of chromium (Cr). Specific examples of the carboxylate of chromium include chromium (II) acetate, chromium (III) acetate, chromium (III)-n-octanoate, chromium (III)-2-ethylhexanoate, chromium (III) benzoate and chromium (III) naphthenate. Of those, chromium (III)-2-ethylhexanoate is particularly preferred.

### (Pyrrole Compound (b))

Specific examples of the pyrrole compound (b) used in the embodiment of the invention include pyrroles such as pyrrole, 2,4-dimethylpyrrole, 2,5-dimethylpyrrole, 2-methyl-5-ethylpyrrole, 2,5-dimethyl-3-ethylpyrrole, 3,4-dimethylpyrrole, 3,4-dichloropyrrole, 2,3,4,5-tetrachloropyrrole, 2-acetylpyrrole and dipyrrole having two pyrrole rings bonded through a substituent, and their derivatives. Examples of the derivative include metal pyrrolide derivatives. Specific examples of the metal pyrrolide derivative include diethylaluminum pyrrolide, ethylaluminum dipyrrolide, aluminum tripyrrolide, sodium pyrrolide, lithium pyrrolide, potassium pyrrolide, diethylaluminum(2,5-dimethylpyrrolide), ethylaluminum bis(2,5-dimethylpyrrolide), aluminum tris(2,5-dimethyl-pyrrolide), sodium(2,5-dimethylpyrrolide), lithium(2,5-dimethylpyrrolide) and potassium(2,5-dimethylpyrrolide). Of those, 2,5-dimethylpyrrole and diethylaluminum(2,5-dimethylpyrrolide) are preferred. (Here, the aluminum pyrrolides are not included in the aluminum-containing compound (c). Furthermore, the halogen-containing pyrrole compound (b) is not included in the halogen-containing compound (d).)

### (Pyrrole Dimer)

The pyrrole dimer in the embodiment of the invention is dipyrrole having a structure represented by the general formula (I), in which two pyrrole rings are directly coupled. The formula (I) shows only a pyrrole ring, and the respective ring may have one or a plurality of hydrogen atoms or hydrocarbon substituents having from 1 to 20 carbon atoms.

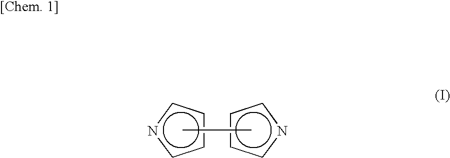

The pyrrole dimer specifically includes 1,1′-dipyrrole coupled with nitrogen atom-nitrogen atom; 1,2′-dipyrrole and 1,3′-dipyrrole, coupled with nitrogen atom-carbon atom; and 2,2′-dipyrrole, 3,3′-dipyrrole and 2,3′-dipyrrole, coupled with carbon-carbon, represented by the formula (II). The general formula (II) shows only a pyrrole ring, and the respective ring may have one or plurality of a hydrogen atoms or hydrocarbon substituents having from 1 to 20 carbon atoms. Depending on the bonding position and the kind of the substituent of the pyrrole compound molecule, atoms at 4- and 5-positions are present on the pyrrole ring, and the general formula (II) encompasses those compounds.

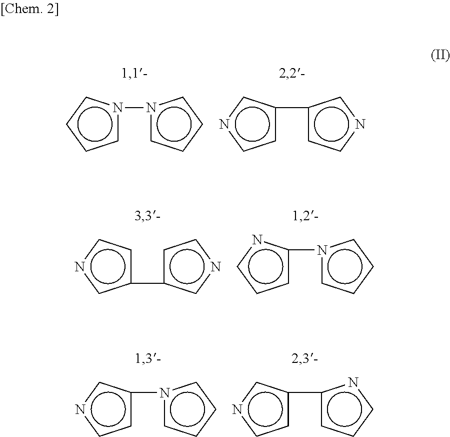

It is generally considered that the pyrrole compound used in the production of an α-olefin low polymer forms the above-described pyrrole dimer by an oxidizing substance present in the system during preparation of the pyrrole compound catalyst, reaction or storage. It is considered that when the dimer thus formed is present exceeding the solubility to a solvent in the system, the dimer converts into an oil component in a solution, separates, and adheres to a reactor wall, an impeller, a nozzle and the like. Furthermore, where the by-produced polymer and the catalyst component are present as a solid in the system, there is a possibility that the dimer incorporates the solid therein, thereby accelerating fouling of a tank wall, a pipe wall, an impeller, an internal coil and the like. As a result, this may give rise to the problem such that heat exchange efficiency of a heat exchanger and a reactor deteriorates, or catalyst feed of from a catalyst preparation tank to a reactor is not stably conducted due to adhesion of a catalyst to the catalyst preparation tank. Additionally, it is considered that economical problems of highly frequent cleaning work, loss of a catalyst, and the like occur.

In the embodiment of the invention, a pyrrole compound having a pyrrole dimer concentration of 2% by weight or less, and preferably 1% by weight or less, is used in the preparation of the chromium series catalyst. Where the concentration of the pyrrole dimer is excessively large, the amount of deposits adhered to a catalyst tank for conducting catalyst preparation and a reactor for obtaining an α-olefin low polymer tends to increase.

The concentration of a pyrrole dimer in a pyrrole compound can be adjusted by purifying the pyrrole compound. In general, the pyrrole compound can be purified with distillation, column purification, adsorption separation and the like. The pyrrole compound is preferably purified with distillation for the reason that complicated operation is less, there is a relatively large difference in boiling point between a pyrrole and its dimer, and as a result, separation is easy.

Where the pyrrole compound is a metal pyrrolide derivative, it is preferred that a pyrrole before derivation is purified with the above-described method, thereafter adjusting the metal pyrrolide derivative.

As the distillation condition, pressure may be freely adjusted from pressurization to reduced pressure, depending on temperature of a heat source that can be used. Where distillation is conducted under reduced pressure, the distillation is preferably conducted at 1 to 300 Torr. When pressure is reduced, it is preferred that joints of an apparatus are sealed with nitrogen or the like, and the possibility of introducing even a slight amount of air is excluded. Furthermore, it is possible to distill while decomposing a pyrrole polymer such as a dimer or a trimer into a monomer by co-existing a reducing agent such as calcium hydride in a distillation apparatus.

The purified pyrrole compound is preferably sealed with an inert gas having an oxygen concentration of 1% or less after purification, and stored under light shielding.

The inert gas used includes nitrogen and argon. Where a gas is used in a large amount, nitrogen gas is preferred. Furthermore, the inert gas used is preferably an inert gas that was deoxidized by the conventional means.

When the purified pyrrole compound is stored, it is preferred that the compound is stored in a stainless steel container or a carbon steel container under light shielding.

In the embodiment of the invention, when the purified pyrrole compound is supplied to a catalyst preparation tank of low polymerization reaction of an α-olefin, it is preferred that a storage tank, piping incidental to the tank, measuring instrument transmission piping and the like are all previously substituted with an inert gas, and the compound is then supplied through the supply piping. After introduction, it is preferred to be under slightly pressurized state with the inert gas.

In general, it is presumed that impurities in the pyrrole compound are present as their dimer, trimer or multimer, and their mixtures. Of the impurities, a dimer is formed in large amount, and is therefore considered to be the main component of impurities. Quantitative analysis can be conducted with an internal reference method by a gas chromatography having a hydrogen flame ionization detector. Because a dimer is composed of plural structures, the quantitative value is calculated with the total value of dimer peaks. The dimer peak on a chromatographic chart can be specified by a general gas chromatograph mass spectrometer, and if necessary, by combining with an atomic emission detector (nitrogen atom)

### (Aluminum-Containing Compound (c))

The aluminum-containing compound (c) used in the embodiment of the invention includes at least one compound such as a trialkylaluminum compound, an alkoxy-alkylaluminum compound and a hydrogenated alkylaluminum compound. Specific examples thereof include trimethyl-aluminum, triethylaluminum, triisobutylaluminum, dimethyl-aluminum monochloride, diethylaluminum ethoxide and diethylaluminum hydride. Of those, triethylaluminum is particularly preferred.

### (Halogen-Containing Compound (d))

The chromium series catalyst used in the embodiment of the invention contains the halogen-containing compound (d) as the fourth component according to need. Examples of the halogen-containing compound (d) include at least one compound of a halogenated alkylaluminum compound, a linear halohydrocarbon having 2 or more carbon atoms and having 3 or more halogen atoms, and a cyclic halohydrocarbon having 3 or more carbon atoms and having 3 or more halogen atoms. (The halogenated alkylaluminum compound is not included in the aluminum-containing compound (c)). Specific examples thereof include diethylaluminum chloride, ethylaluminum sesquichloride, carbon tetrachloride, 1,1,1-trichloroethane, 1,1,2,2-tetrachloroethane, pentachloroethane, hexachloroethane, 1,2,3-trichlorocyclopropane, 1,2,3,4,5,6-hexachlorocyclo-hexane and 1,4-bis(trichloro-methyl)-2,3,5,6-tetrachloro-benzene.

In the embodiment of the invention, the polymerization of an α-olefin is preferably that the chromium compound (a) and the aluminum-containing compound (c) are not previously contacted, or an α-olefin and the chromium series catalyst are previously contacted in a state that the previous contact time is short. Such a contact embodiment makes it possible to selectively conduct trimerization reaction of ethylene, thereby obtaining 1-hexene from ethylene as a raw material in high yield.

The contact embodiment in the above continuous reaction system specifically includes the following (1) to (9).

(1) A method of simultaneously introducing a mixture of the catalyst components (a), (b) and (d) and the catalyst component (c) into a reactor, respectively.

(2) A method of simultaneously introducing a mixture of the catalyst components (b) to (d) and the catalyst component (a) into a reactor, respectively.

(3) A method of simultaneously introducing a mixture of the catalyst components (a) and (b) and a mixture of the catalyst components (c) and (d) into a reactor, respectively.

(4) A method of simultaneously introducing a mixture of the catalyst components (a) and (d) and a mixture of the catalyst components (b) and (c) into a reactor, respectively.

(5) A method of simultaneously introducing a mixture of the catalyst components (a) and (b), catalyst component (c) and the catalyst component (d) into a reactor, respectively.

(6) A method of simultaneously introducing a mixture of the catalyst components (c) and (d), catalyst component (a) and the catalyst component (b) into a reactor, respectively.

(7) A method of simultaneously introducing a mixture of the catalyst components (a) and (d), catalyst component (b) and the catalyst component (c) into a reactor, respectively.

(8) A method of simultaneously introducing a mixture of the catalyst components (b) and (c), catalyst component (a) and the catalyst component (d) into a reactor, respectively.

(9) A method of simultaneously and independently introducing each of the catalyst components (a) to (d).

The above-described each catalyst component is generally dissolved in a solvent used in the reaction, and supplied to a reactor.

The “embodiment that the chromium compound (a) and the aluminum-containing compound (c) are not previously contacted” is not limited to the initiation time of the reaction, and means that such an embodiment is maintained even in the supply of the subsequent additional α-olefin and catalyst components into the reactor.

Furthermore, in a batch reaction type, it is desired that the same embodiment is utilized.

The ratio of each constituent in the chromium series catalyst used in the embodiment of the invention is generally that the pyrrole compound (b) is from 1 to 50 moles, and preferably from 1 to 30 moles, per mole of the chromium compound (a), and the aluminum-containing compound (c) is from 1 to 200 moles, and preferably from 10 to 150 moles, per mole of the chromium compound. When the halogen-containing compound (d) is contained in the chromium series catalyst, the halogen-containing compound (d) is from 1 to 50 moles, and preferably from 1 to 30 moles, per mole of the chromium compound (a).

In the embodiment of the invention, the amount of the chromium series catalyst used is not particularly limited, but is generally from 1.0×10⁻⁷ to 0.5 mole, preferably from 5.0×10⁻⁷ to 0.2 mole, and further preferably from 1.0×10⁻⁶ to 0.05 mole, in terms of chromium atom of the chromium compound (a) per 1 liter of the solvent described hereinafter.

By using such a chromium series catalyst, for example when ethylene is used as a raw material, hexene which is a trimer of ethylene can be obtained in selectivity of 90% or more. In this case, the proportion of 1-hexene occupied in hexene can be 99% or more.

### (Solvent)

In the production method of an α-olefin low polymer to which the embodiment of the invention is applied, the reaction of an α-olefin can be conducted in a solvent.

Such a solvent is not particularly limited. However, for example, chain saturated hydrocarbons or alicyclic saturated hydrocarbons, having from 1 to 20 carbon atoms, such as butane, pentane, 3-methylpentane, hexane, heptane, 2-methylhexane, octane, cyclohexane, methylcyclohexane, 2,2,4-trimethylpentane and decalin; and aromatic hydrocarbons such as benzene, toluene, xylene, ethylbenzene, mesitylene and tetralin are used. Furthermore, an α-olefin low polymer may be used as a solvent. Those can be used alone or as a mixed solvent.

In particular, the preferred solvent is chain saturated hydrocarbons or alicyclic saturated hydrocarbons, having from 4 to 10 carbon atoms. When those solvents are used, by-produced polymers such as a polyethylene can be suppressed. Furthermore, when the alicyclic saturated hydrocarbons are used, high catalyst activity tends to be obtained.

### (Production Method of α-Olefin Low Polymer)

The α-olefin low polymer in the present invention means an oligomer comprising a plurality of an α-olefin as a monomer being bonded. Specifically, it means a polymer comprising 2 to 10 of an α-olefin as a monomer being bonded.

The production method of an α-olefin low polymer is described by referring to an example of the production of 1-hexene which is a trimer of ethylene as an α-olefin low polymer using ethylene as an α-olefin.

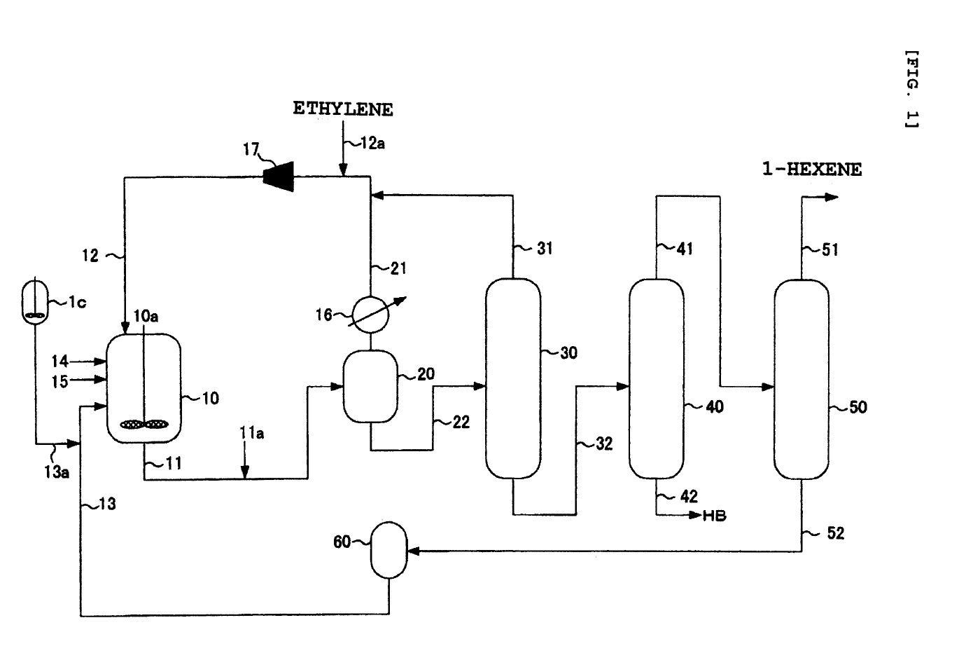

FIG. 1 is a view explaining a production flow example of an α-olefin low polymer in the embodiment of the invention. The production flow example of 1-hexene using ethylene as a raw material shown in FIG. 1 shows a completely mixing and stirring type reactor 10 in which ethylene is subjected to low polymerization in the presence of a chromium series catalyst, a degassing tank 20 that separates an unreacted ethylene gas from a reaction liquid withdrawn from the reactor 10, an ethylene separation column 30 that distills ethylene in the reaction liquid withdrawn from the degassing tank 20, a high boiling separation column 40 that separates a high boiling substance (hereinafter referred to as “HB” (high boiler)) in the reaction liquid withdrawn from the ethylene separation column 30, and a hexene separation column 50 that distills the reaction liquid withdrawn from the top of the high boiling separation column 40 to distill away 1-hexene.

Furthermore, a compressor 17 that circulates an unreacted ethylene separated in the degassing tank 20 and the condenser 16 into the reactor 10 via a circulation piping 21 is provided.

In FIG. 1, the reactor 10 includes conventional reactors equipped with a stirring machine 10a, baffle, jacket and the like. As the stirring machine 10a, a stirring blade of the type such as paddle, pfaudler, propeller, turbine or the like is used in a combination with a baffle such as a planar plate, a cylinder or a hairpin coil.

As shown in FIG. 1, ethylene is continuously supplied to the reactor 10 from an ethylene supply piping 12a via a compressor 17 and the first supply piping 12. Where the compressor 17 is, for example, two-stage compression system, a circulation piping 31 is connected to the first stage, and a circulation piping 21 is connected to the second stage, thereby making it possible to reduce electricity consumption. Furthermore, a solvent used in low polymerization reaction of ethylene is supplied to the reactor 10 from the second supply piping 13.

On the other hand, the chromium compound (a) and the pyrrole compound (b) previously prepared in the catalyst tank 1c are supplied to the reactor 10 from the second supply piping 13 via a catalyst supply piping 13a, the aluminum-containing compound (c) is supplied from the third supply piping 14, and the halogen-containing compound (d) is supplied from the fourth supply piping 15. Here, the halogen-containing compound (d) may be supplied to the reactor 10 from the second supply piping 13 via a supply piping. Furthermore, in the event that the aluminum-containing compound (c) is supplied to the reactor within several minutes of the contact time with the chromium compound (a), it may be supplied to the reactor 10 from the second supply piping 13 via a supply piping. In employing this system, when a static mixer or the like is provided between the second piping 13 and the reactor, a uniform mixture of each catalyst component can be supplied to the reactor, and as a result, stirring power of the reactor can be reduced.

In the embodiment of the invention, the reaction temperature in the reactor 10 is generally from 0 to 250° C., preferably from 50 to 200° C., and more preferably from 80 to 170° C.

The reaction pressure is in a range of generally from normal pressures to 250 kgf/cm², preferably from 5 to 150 kg/cm², and more preferably from 10 to 100 kgf/cm².

The trimerization reaction of ethylene is preferably conducted such that a molar ratio of 1-hexene to ethylene in the reaction liquid ((molar concentration of 1-hexene in reaction liquid)/(molar concentration of ethylene in reaction liquid)) is from 0.05 to 1.5, and particularly from 0.10 to 1.0. Specifically, it is preferred that in the case of a continuous reaction, a catalyst concentration, reaction pressure and other conditions are adjusted such that the molar ratio of 1-hexene to ethylene in the reaction liquid is in the above range, and in the case of a batchwise reaction, the reaction is stopped at the time that the molar ratio is in the above range. This has the tendency that by-production of components having a boiling point higher than that of 1-hexene is suppressed, thereby further increasing selectivity of 1-hexene.

The reaction liquid continuously withdrawn from the bottom of the reactor 10 via a piping 11 is that trimerization reaction of ethylene is stopped by a deactivator supplied from a deactivator supply piping 11a, and such a reaction liquid is supplied to the degassing tank 20. In the degassing tank 20, unreacted ethylene is degassed from the top thereof, and circulated and supplied to the reactor 10 via the circulation piping 21, the condenser 16, the compressor 17 and the first supply piping 12. The reaction liquid from which unreacted ethylene has been degassed is withdrawn from the bottom of the degassing tank 20.

Operation conditions of the degassing tank 20 are that the temperature is generally from 0 to 250° C., and preferably from 50 to 200° C., and the pressure is generally from normal pressures to 150 kgf/cm², and preferably from normal pressures to 90 kgf/cm².

Subsequently, the reaction liquid from which unreacted ethylene gas has been degassed in the degassing tank 20 is withdrawn from the bottom of the degassing tank 20, and supplied to an ethylene separation column 30 by a piping 22. In the ethylene separation column 30, ethylene is distilled away from the column top by distillation, and circulated and supplied to the reactor 10 via the circulation piping 31 and the first supply piping 12. The reaction liquid from which ethylene has been removed is withdrawn from the bottom.

Operation conditions of the ethylene separation column 30 are that the top pressure is generally from normal pressures to 30 kgf/cm², and preferably from normal pressures to 20 kgf/cm², and the reflux ratio (R/D) is generally from 0 to 500, and preferably from 0.1 to 100.

The reaction liquid from which ethylene has been distilled away in the ethylene separation column 30 is withdrawn from the bottom of the ethylene separation column 30, and supplied to a high boiling separation column 40 by a piping 32. In the high boiling separation column 40, components with high boiling point (HB: high boiler) are withdrawn from the bottom. A distillate from which high boiling components have been separated is withdrawn from the top by a piping 42.

Operation conditions of the high boiling separation column 40 are that the top pressure is generally from 0.1 to 10 kgf/cm², and preferably from 0.5 to 5 kgf/cm², and the reflux ratio (R/D) is generally from 0 to 100, and preferably from 0.1 to 20.

Subsequently, the reaction liquid withdrawn as a distillate from the top of the high boiling separation column 40 is supplied to a hexene separation column 50 by the piping 41. In the hexene separation column 50, 1-hexene is distilled away by distillation from the top by a piping 51. Heptane is withdrawn from the bottom of a hexene separation column 50, and stored in a solvent drum 60 via a solvent circulation piping 52, and circulated and supplied as a reaction solvent to the reactor 10 via the second supply piping 13.

Operation conditions of the hexene separation column 50 are that the top pressure is generally from 0.1 to 10 kgf/cm², and preferably from 0.5 to 5 kgf/cm², and the reflux ratio (R/D) is generally from 0 to 100, and preferably from 0.1 to 20.

### EXAMPLES

The present invention is described further specifically based on the examples. However, the present invention is not limited to the following examples so far as it does not depart from its gist.

### Examples 1 to 3, and Comparative Example 1

A chromium series catalyst was prepared using pyrrole(2,5-dmethylpyrrole) containing a pyrrole dimer having a given concentration shown in Table 1.

Dehydrated heptane (150 ml) was charged in a glass-made three-necked flask having a stirring machine, and 2,5-dimethylpyrrole (0.016 mol) having a given purity shown in Table 1 and triethylaluminum (0.016 mol) were added thereto.

Temperature of the flask was elevated, and reflux of heptane was conducted for 3 hours at normal pressures. Thereafter, the flask was cooled to 80° C.

Subsequently, 0.0027 mol of chromium 2-ethyl-hexanoate was added, followed by heating at 80° C. for 30 minutes.

Thereafter, the flask was cooled to room temperature, a solution in the flask was separated by decantation, and the flask was dried at 150° C. for 8 hours.

Next, the amount of deposits in the flask used for catalyst preparation was measured. The results are shown in Table 1.

The amount of deposits is expressed as the proportion of weight of deposits to the total weight of chromium 2-ethylhexanoate, triethylethyl aluminum and 2,5-dimethylpyrrole used for the preparation of a chromium series catalyst.

The concentration of a pyrrole dimer was obtained by an internal reference method (p-xylene) by conducting GC analysis.

TABLE 1

| | | Concentration of | Amount of |
|---|---|---|---|
| | | pyrrole dimer | deposit |
| | | (% by weight) | (%) |
| Example | 1 | 1.30 | 6.46 |
| | 2 | 0.75 | 8.64 |
| | 3 | 0.20 | 8.47 |
| Comparative | 1 | 5.30 | 23.2 |
| Example | | | |

It is seen from the results shown in Table 1 that when a chromium series catalyst was prepared using 2,5-dimethylpyrrole having a concentration of a pyrrole dimer of 2% by weight or less (Examples 1 to 3), the amount of deposits in the flask used for catalyst preparation is smaller. Therefore, when those pyrrole compounds are used as a catalyst component in producing an α-olefin low polymer, the amount of deposits adhered to a reactor wall, an impeller, a nozzle and the like is reduced, and it is expected that a tank wall, a pipe wall, an impeller, an internal coil and the like can be prevented from fouling. Furthermore, the effects can be expected such that heat exchange efficiency of a heat exchanger and a reactor is prevented from being decreased, and catalyst feed of from a catalyst preparation tank to a reactor can stably be conducted.

On the other hand, when the concentration of the pyrrole dimer exceeds 2% by weight (Comparative Example 1), it is seen that the amount of deposits in the flask used for catalyst preparation is increased.

### Examples 4 to 6, and Reference Example 1

A reagent purchased was distilled and purified to obtain 2,5-dimethylpyrrole having a purity of 99.80%. The remaining component is a pyrrole dimer (concentration: 0.2% by weight).

2,5-Dimethylpyrrole was stored under an oxygen concentration shown in Table 2 and under given conditions, and concentration change of a pyrrole dimer was measured.

2,5-Dimethylpyrrole was stored by the following operation.

Nitrogen and air were mixed to make up a nitrogen gas having a given oxygen concentration. 5 ml of 2,5-dimethylpyrrole after distillation (purity 99.80%) was charged in three way cock test tube substituted with nitrogen.

Make-up nitrogen gas previously prepared to have a given oxygen concentration was sampled, and it was confirmed to be a given concentration using Teledyne oxygen analyzer (trace oxygen analyzer). Thereafter, the nitrogen gas was introduced into the test tube through three way cock. A gas phase part (50 ml) was substituted with the make-up gas, and the test tube was allowed to stand at room temperature at dark place.

During allowing to stand, gas substitution of the gas phase part of the test tube was repeated every 12 hours. 2,5-Dimethylpyrrole was sampled every given period of time shown in Table 2, and the concentration of a pyrrole dimer was analyzed with GC. The concentration was obtained by an internal reference method (p-xylene).

The results are shown in Table 2.

TABLE 2

| | | | | | | Reference |
|---|---|---|---|---|---|---|
| | | | Example | | | Example |
| | | | 4 | 5 | 6 | 1 |
| Oxygen Concentration (%) | | | 0 | 0.1 | 1 | 21 |
| Concentration | Time (hr) | 0 | 0.20 | 0.20 | 0.20 | 0.20 |
| of pyrrole | | 72 | 0.19 | 0.17 | 0.11 | 0.89 |
| dimer | | 144 | 0.34 | 0.68 | 0.58 | 2.13 |
| (% by weight) | | 298 | 0.62 | 1.06 | 1.60 | 3.34 |

It is seen from the results shown in Table 2 that when 2,5-dimethylpyrrole is, after purification, sealed with a nitrogen gas having an oxygen concentration of 1% or less, and stored under light shielding (Examples 4 to 6), the concentration of the pyrrole dimer is maintained at 2% by weight or less. Therefore, when those pyrrole compounds are used as a catalyst component in producing an α-olefin low polymer, the amount of deposits adhered to a reactor wall, an impeller, a nozzle and the like is reduced, and it is expected that a tank wall, a pipe wall, an impeller, an internal coil and the like can be prevented from fouling. Furthermore, the effects can be expected such that heat exchange efficiency of a heat exchanger and a reactor is prevented from being decreased, and catalyst feed of from a catalyst preparation tank to a reaction can stably be conducted.

On the other hand, when 2,5-dimethylpyrrole is, after purification, sealed with a nitrogen gas having an oxygen concentration of 21% (air) (Reference Example 1) it is seen that the pyrrole dimer is increased.

While the invention has been described in detail and with reference to the specific embodiments thereof, it will be apparent to one skilled in the art that various changes and modifications can be made therein without departing from the spirit and scope thereof.

This application is based on Japanese Patent Application (Patent Application No. 2006-354249) filed Dec. 28, 2006, the entire contents thereof being hereby incorporated by reference.

### INDUSTRIAL APPLICABILITY

According to the present invention, in the production of an α-olefin low polymer, deposits adhered to a catalyst preparation tank and a rector are reduced, and running operation can be stabilized. Therefore, the industrial value of the present invention is remarkable.

1. A production method of an α-olefin low polymer, which comprises polymerizing an α-olefin in the presence of a chromium series catalyst, characterized in that:
the chromium series catalyst is constituted of at least a chromium compound (a), a pyrrole compound (b) and an aluminum-containing compound (c), and
a concentration of a pyrrole dimer contained in the pyrrole compound (b) in the chromium series catalyst that is applied to the low polymerization reaction of the α-olefin is 2% by weight or less based on the pyrrole compound (b).

2. The production method of an α-olefin low polymer as claimed in claim 1, characterized in that the pyrrole compound (b) is sealed with an inert gas having an oxygen concentration of 1% or less after purification, and stored under light shielding.

3. The production method of an α-olefin low polymer as claimed in claim 1, characterized in that the pyrrole compound (b) is a pyrrole having one or plural alkyl group having from 1 to 4 carbon atoms.

4. The production method of an α-olefin low polymer as claimed in claim 1, characterized in that the chromium series catalyst is constituted of a combination of the chromium compound (a), the pyrrole compound (b), the aluminum-containing compound (c) and a halogen-containing compound (d).

5. The production method of an α-olefin low polymer as claimed in claim 1, characterized in that the α-olefin is ethylene.

6. The production method of an α-olefin low polymer as claimed in claim 1, characterized in that the α-olefin low polymer is 1-hexene.

7. A storage method of a pyrrole compound, characterized in that a concentration of a pyrrole dimer in the pyrrole compound is 2% by weight or less based on the pyrrole compound.

8. The storage method as claimed in claim 7, characterized in that the pyrrole compound is purified, and then sealed with an inert gas having an oxygen concentration of 1% or less under light shielding.
